(19) United States
(12) Patent Application Publication
(10) Pub. No.: US 2023/0139371 A1
(43) Pub. Date: May 4, 2023

(54) LOW-FREQUENCY TREATMENT APPARATUS, CONTROL METHOD, AND NON-TRANSITORY RECORDING MEDIUM IN WHICH CONTROL PROGRAM IS STORED

(71) Applicant: OMRON HEALTHCARE Co., Ltd., Kyoto (JP)

(72) Inventors: Kosuke INOUE, Kyoto (JP); Tomoyuki NISHIDA, Kyoto (JP); Kohei HAYAKAWA, Kyoto (JP); Yoshikazu INAMI, Kyoto (JP); Kansuke FUKUDA, Kyoto (JP); Yoshito ASAI, Kyoto (JP); Naoki TAKEISHI, Kyoto (JP); Gaku HASEGAWA, Kyoto (JP); Mayumi AKATSUKA, Kyoto (JP); Ryo MIZUYAMA, Kyoto (JP); So NOGUCHI, Osaka (JP)

(21) Appl. No.: 18/148,811

(22) Filed: Dec. 30, 2022

Related U.S. Application Data
(63) Continuation of application No. PCT/JP2021/022945, filed on Jun. 16, 2021.

(30) Foreign Application Priority Data
Jul. 6, 2020 (JP) 2020-116599

(51) Int. Cl. A61N 1/36; A61N 1/04; A61N 5/06

(52) U.S. Cl. CPC A61N 1/36034; A61N 1/0452; A61N 1/0492; A61N 5/0613; A61N 2005/0626; A61N 2005/0663

(57) ABSTRACT

Provided is a technique that can improve a physical sensation of a user during treatment. A low-frequency treatment device according to one aspect of the present invention includes a pad portion configured to be able to supply a low-frequency pulse current to a contact target, and an output unit configured to output, during supply of the low-frequency pulse current by the pad portion, at least one of light and sound synchronized with a change in a supply state of the low-frequency pulse current. For example, the low-frequency treatment device according to one aspect of the present invention includes a control unit configured to perform control of supply of the low-frequency pulse current by the pad portion and control, in conjunction with the control of supply of the low-frequency pulse current, of output of at least one of the light and sound by the output unit.

### CROSS-REFERENCE TO RELATED APPLICATIONS

This application is the U.S. national stage application filed pursuant to 35 U.S.C. 365(c) and 120 as a continuation of International Patent Application No. PCT/JP2021/022945, filed Jun. 16, 2021, which application claims priority to Japanese Patent Application No. 2020-116599, filed Jul. 6, 2020, which applications are incorporated herein by reference in their entireties.

### TECHNICAL FIELD

The present invention relates to a low-frequency treatment device, a control method, and a non-transitory recording medium in which a control program is stored.

### BACKGROUND ART

Conventionally, a low-frequency treatment device is known that performs treatment of, for example, relieving shoulder stiffness of a user by attaching a pad including a conductive layer to the body of the user and supplying a low-frequency pulse current to the body.

Patent Document 1 describes that power is supplied to a circuit that generates electrical stimulation or heat and that the completion of preparation for applying stimulation to the body of a human or an animal by the circuit is indicated by light-emitting diodes.

Patent Document 2 describes a configuration in which light-emitting diodes on pads selected by a user are lighted up to assist the user to attach the proper pads to proper regions of the body before the user finishes properly attaching the pads to the body.

### CITATION LIST

### Patent Literature

Patent Document 1: JP 2015-514460 T
Patent Document 2: JP 2013-540543 T

### SUMMARY OF INVENTION

### Technical Problem

While the effect of treatment, for example, relieving shoulder stiffness with a low-frequency pulse current has been accepted, the effect of treatment is also affected by a physical sensation of a user. Therefore, it is believed that improvement of the physical sensation of the user during treatment with the low-frequency pulse current is effective to improve the effect of treatment.

Further, in order to prevent muscle atrophy that may cause shoulder stiffness and the like, it is desirable to suppress worsening of symptoms by frequently performing treatment with the low-frequency pulse current. For such purpose, it is predictably effective to improve user’s motivation for treatment with the low-frequency pulse current while improving the physical sensation of the user during treatment with the low-frequency pulse current.

However, in the conventional technology, a physical sensation of a user during treatment depends only on electrical stimulation to the skin and muscles, which is produced by the voltage, frequency, or the like of the low-frequency pulse current, and it was difficult to improve the physical sensation of the user during treatment. Furthermore, no means for solving such a problem is disclosed in Patent Documents 1 or 2.

The present invention is made in view of such circumstances in one aspect, and an object of the present invention is to provide a technology that can improve a physical sensation of a user during treatment.

### Solution to Problem

The present invention adopts the following configurations to solve the above-described problems.

In other words, a low-frequency treatment device according to one aspect of the present invention includes a pad portion configured to be able to supply a low-frequency pulse current to a contact target, and an output unit configured to outputs, during supply of the low-frequency pulse current by the pad portion, at least one of light and sound synchronized with a change in a supply state of the low-frequency pulse current.

In the configuration described above, the light or sound synchronized with a change in a supply state of the low-frequency pulse current is output, and thus a complex stimulation of electrical stimulation by the low-frequency pulse current and stimulation different from the electrical stimulation (at least one of visual sensation and auditory sensation) can be applied to a user. As a result, as compared to a configuration in which electrical stimulation by the low-frequency pulse current is singly applied to a user, the electrical stimulation by the low-frequency pulse current is effectively perceived by the user, and thus a physical sensation for treatment by the low-frequency pulse current can be improved.

In the low-frequency treatment device according to the aspect described above, for example, a control unit is provided that is configured to perform control of supply of the low-frequency pulse current by the pad portion and control, in conjunction with the control of supply of the low-frequency pulse current, of output of at least one of the light and sound by the output unit. According to the configuration, the light or sound synchronized with a change in a supply state of the low-frequency pulse current can be output.

In the low-frequency treatment device according to the aspect described above, a supply state of the low-frequency pulse current includes, for example, stimulation intensity by the low-frequency pulse current to the contact target. According to the configuration, the light or sound synchronized with the stimulation intensity by the low-frequency pulse current is output, and thus electrical stimulation by the low-frequency pulse current can be effectively perceived by the user.

In the low-frequency treatment device according to the aspect described above, for example, the stimulation intensity includes a frequency of the low-frequency pulse current.

In the low-frequency treatment device according to the aspect described above, for example, the stimulation intensity includes a voltage of the low-frequency pulse current.

In the low-frequency treatment device according to the aspect described above, for example, the stimulation intensity includes a pulse width of the low-frequency pulse current.

In the low-frequency treatment device according to the aspect described above, for example, a supply state of the low-frequency pulse current includes a supply position of the low-frequency pulse current in the pad portion. According to the configuration, the light or sound synchronized with a change of the supply position of the low-frequency pulse current is output, and electrical stimulation by the low-frequency pulse current can be effectively perceived by the user.

In the low-frequency treatment device according to the aspect described above, the output unit may be disposed in the pad portion. According to the configuration, both electrical stimulation and stimulation different from the electrical stimulation are output from the pad portion, and thus the user can easily recognize that the both stimulations are synchronized. Thus, the electrical stimulation is more effectively perceived by the user, and thus a physical sensation for treatment by the low-frequency pulse current can be improved.

In the low-frequency treatment device according to the aspect described above, for example, the pad portion includes an electrode configured to be able to supply the low-frequency pulse current, and the output unit is disposed at a position corresponding to the position of the electrode in the pad portion, and includes a light emitting unit configured to emit light in synchronization with supply of the low-frequency pulse current by the electrode. According to the configuration, both electrical stimulation and visual stimulation are output from each position to which the both stimulations correspond, and thus the user can easily recognize that the both stimulations are synchronized. Thus, the electrical stimulation is more effectively perceived by the user, and thus a physical sensation for treatment by the low-frequency pulse current can be improved. Note that the position corresponding to the position of the electrode is a position close to the electrode, and is, for example, a position closest to the electrode in the surface, which can be seen from the user, of the pad portion attached to the user.

In the low-frequency treatment device according to the aspect described above, the pad portion may include a plurality of electrodes configured to be able to supply the low-frequency pulse current, and the output unit may be disposed at a position corresponding to the position of each of the plurality of electrodes in the pad portion, and may include a plurality of light emitting units configured to respectively emit light in synchronization with supply of the low-frequency pulse current by the corresponding electrodes. According to the configuration, the light emitting position changes with a change of the position to which the low-frequency pulse current is supplied. Thus, the electrical stimulation is more effectively perceived by the user, and thus a physical sensation for treatment by the low-frequency pulse current can be improved.

In the low-frequency treatment device according to the aspect described above, the pad portion includes, on a surface opposite to the contact target, an insulating layer covering the electrode, and the insulating layer includes a transparent member configured to diffuse light of the light emitting unit. According to the configuration, for example, even when the light emitting unit is small, an insulating film is made to emit light, and impressive visual stimulation can be applied to the user.

In the low-frequency treatment device according to the aspect described above, the electrode is formed of a transparent conductive film configured to diffuse light of the light emitting unit. According to the configuration, the transparent conductive film of the electrode is also made to emit light in addition to the insulating film, and impressive visual stimulation can be applied to the user.

In the low-frequency treatment device according to the aspect described above, for example, the output unit is configured to output light changing at least one of the color, brightness, and area in synchronization with a change in a supply state of the low-frequency pulse current.

In the low-frequency treatment device according to the aspect described above, the output unit may be configured to output light having color that changes depending on at least one of intensity and waveform of the low-frequency pulse current. According to this configuration, the difference in treatment content according to the intensity or waveform of the low-frequency pulse current is visually emphasized to be perceived by the user, and thus a physical sensation for treatment by the low-frequency pulse current can be improved.

In the low-frequency treatment device according to the aspect described above, for example, the output unit is configured to output sound with changing at least one of the sound pressure, pitch, and tone color in synchronization with a change in a supply state of the low-frequency pulse current.

In the low-frequency treatment device according to the aspect described above, the output unit may be configured to output sound having the tone color that changes depending on at least one of intensity and waveform of the low-frequency pulse current. According to this configuration, the difference in treatment content according to the intensity or waveform of the low-frequency pulse current is visually emphasized to be perceived by the user, and thus a physical sensation for treatment by the low-frequency pulse current can be improved.

A control method according to one aspect of the present invention is a control method of a low-frequency treatment device including a pad portion configured to be able to supply a low-frequency pulse current to a contact target, and an output unit, the control method including controlling the pad portion to supply the low-frequency pulse current, and controlling the output unit to output, during supply of the low-frequency pulse current by the pad portion, at least one of light and sound synchronized with a change in a supply state of the low-frequency pulse current.

A control program according to one aspect of the present invention is a control program of a low-frequency treatment device including a pad portion configured to be able to supply a low-frequency pulse current to a contact target, and an output unit, the control program allowing a computer to execute controlling the pad portion to supply the low-frequency pulse current, and controlling the output unit to output, during supply of the low-frequency pulse current by the pad portion, at least one of light and sound synchronized with a change in a supply state of the low-frequency pulse current.

### Advantageous Effects of Invention

According to the present invention, the technology can be provided that can improve a physical sensation of a user during treatment.

### BRIEF DESCRIPTION OF DRAWINGS

FIG. 1 is a drawing illustrating a low-frequency treatment device 10 that is an application example of the present invention.

FIG. 2 is a perspective view illustrating the configuration of a low-frequency treatment device 20 that is an example of the low-frequency treatment device 10.

FIG. 3 is an exploded perspective view illustrating a state where a pad portion 2 and a main body portion 3 of the low-frequency treatment device 20 are separated.

FIG. 4 is a plan view illustrating the pad portion 2 of the low-frequency treatment device 20.

FIG. 5 is a bottom view illustrating the pad portion 2 of the low-frequency treatment device 20.

FIG. 6 is a block diagram illustrating an example of the hardware configuration of the main body portion 3.

FIG. 7 is a diagram illustrating an example of a pulse voltage applied by the main body portion 3 to a pad 21.

FIG. 8 is a cross-sectional view illustrating an example of the pad 21 including a light emitter.

FIG. 9 is a diagram illustrating an example of the pad 21 having a multiple electrode structure.

FIG. 10 is a diagram illustrating an example of light emitting in a case where the pad 21 has the multiple electrode structure.

FIG. 11 is a diagram illustrating an example of the low-frequency treatment device 20 that outputs sound synchronized with a low-frequency pulse current.

FIG. 12 is a diagram illustrating the configuration of a low-frequency treatment device 200 that is another example of the low-frequency treatment device 10.

### DESCRIPTION OF EMBODIMENTS

Embodiments according to an aspect of the present invention will be described below based on the drawings.

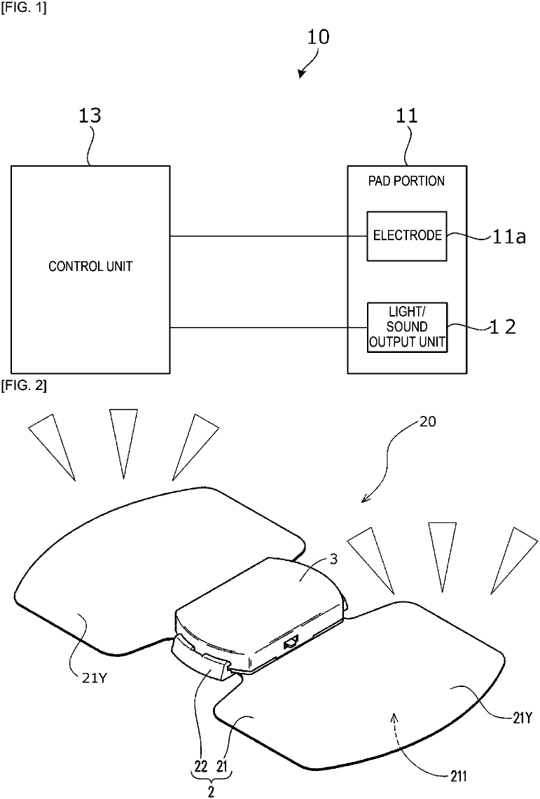

Application Example FIG. 1 is a diagram illustrating the low-frequency treatment device 10 that is an application example of the present invention.

The low-frequency treatment device 10 is a device configured to perform treatment or the like on a user by supplying a low-frequency pulse current and applying electrical stimulation to the user. The frequency of the low-frequency pulse current is, for example, approximately 1 to 1200 Hz. The low-frequency treatment device 10 includes a pad portion 11, a control unit 13, and a light/sound output unit 12.

The pad portion 11 can be attached to the body of the user. Also, the pad portion 11 includes an electrode 11a, and can supply a low-frequency pulse current from the electrode 11a to the body of the user, which is an attachment location (a contact target). The supply of the low-frequency pulse current by the pad portion 11 is controlled by the control unit 13.

The light/sound output unit 12 is an output unit that outputs, during supply of the low-frequency pulse current by the pad portion 11, at least one of light and sound synchronized with a change in a supply state of the low-frequency pulse current. For example, the light/sound output unit 12 includes at least one of a light emitting unit and a sound emitting unit. The output of light or sound output by the light/sound output unit 12 is controlled by the control unit 13. Synchronization is, for example, interlocking, i.e., changing at the same time.

The light emitting unit is a portion that appears to be luminous as viewed with the user’s eyes, and is, for example, a light source such as a light emitting diode (LED). Also, the light emitting unit may be a combination of a light source and a light guiding member that guides light of the light source.

The sound emitting unit is a portion that emits sound audible to the user, and is, for example, a speaker that converts an electrical signal to sound. Also, the sound emitting unit may be a combination of a speaker and a transmission member that transmits sound of the speaker.

The light synchronized with a change in a supply state of the low-frequency pulse current is, for example, light changing at least one of the color, brightness, and area in synchronization with a change in a supply state of the low-frequency pulse current. The area of light is the area of a portion that emits light. For example, the light/sound output unit 12 is a light emitting unit that emits light, at least one of the color, brightness, and area of which is proportional to the stimulation intensity of the low-frequency pulse current. As an example, in accordance with the control by the control unit 13, the light/sound output unit 12 emits light when the low-frequency pulse current is supplied, and does not emit light when the low-frequency pulse current is not supplied.

The sound synchronized with a change in a supply state of the low-frequency pulse current is, for example, sound changing at least one of the sound pressure, pitch, and tone color in synchronization with a change in a supply state of the low-frequency pulse current. For example, the light/sound output unit 12 is a sound emitting unit that outputs sound, at least one of the sound pressure, pitch, and tone color of which is proportional to the stimulation intensity of the low-frequency pulse current. As an example, in the light/sound output unit 12, in accordance with the control by the control unit 13, the sound is produced when the low-frequency pulse current is supplied, and the sound is not produced when the low-frequency pulse current is not supplied.

The control unit 13 performs a first control for controlling supply of the low-frequency pulse current by the pad portion 11. Further, the control unit 13 performs a second control for controlling output of at least one of light and sound by the light/sound output unit 12.

In addition, the control unit 13 performs the first control and the second control in conjunction with each other. In other words, the control unit 13 performs the second control based on the content of the first control or performs the first control based on the content of the second control. Thus, the light or sound that is synchronized with a change in a supply state of the low-frequency pulse current by the pad portion 11 can be output from the light/sound output unit 12.

As the first control, for example, the control unit 13 generates a pulse voltage waveform for supplying the low-frequency pulse current by the electrode 11a and applies the generated pulse voltage waveform to the electrode 11a. In addition, as the second control, the control unit 13 directly outputs the generated pulse voltage waveform to the light/sound output unit 12 as a drive signal for allowing the light/sound output unit 12 to output light or sound, or adds processing such as voltage conversion to the generated pulse voltage waveform and then outputs the pulse voltage waveform to the light/sound output unit 12 as a drive signal for allowing the light/sound output unit 12 to output light or sound.

Note that a method for performing the first control and the second control in conjunction with each other is not limited to the foregoing method. For example, the control unit 13 may separately generate a pulse voltage waveform for supplying the low-frequency pulse current by the electrode 11a and a drive signal for allowing the light/sound output unit 12 to output light or sound, and output the generated pulse voltage waveform and the generated drive signal respectively to the electrode 11a and the light/sound output unit 12.

As just described, the low-frequency treatment device 10 outputs, from the light/sound output unit 12, light or sound synchronized with a change in a supply state of the low-frequency pulse current. Thus, a complex stimulation of electrical stimulation by the low-frequency pulse current and stimulation different from the electrical stimulation (at least one of visual sensation and auditory sensation) can be applied to the user. As a result, as compared to a configuration in which electrical stimulation by the low-frequency pulse current is singly applied to a user, the electrical stimulation by the low-frequency pulse current is effectively perceived by the user, and thus a physical sensation for treatment by the low-frequency pulse current can be improved.

A supply state of the low-frequency pulse current described above includes, for example, stimulation intensity to the user by the low-frequency pulse current supplied by the pad portion 11. In other words, the pad portion 11 can adjust the stimulation intensity to the user by the low-frequency pulse current to be supplied, and the light/sound output unit 12 outputs at least one of light and sound synchronized with a change in stimulation intensity to the user by the low-frequency pulse current supplied by the pad portion 11.

Additionally, a supply state of the low-frequency pulse current described above includes, for example, a supply position of the low-frequency pulse current in the pad portion 11. In other words, the pad portion 11 can supply the low-frequency pulse current from a plurality of positions in a region in which the pad portion 11 is brought into contact with the user, and the light/sound output unit 12 outputs at least any one of light and sound synchronized with a change of the position to which the pad portion 11 supplies the low-frequency pulse current.

The stimulation intensity of the low-frequency pulse current includes, for example, at least one of a frequency (i.e., a period) of the low-frequency pulse current, a voltage of the low-frequency pulse current, and a pulse width of the low-frequency pulse current. For example, the higher the frequency of the low-frequency pulse current is, the shorter an interval of stimulation is, and thus the stimulation has high intensity. Further, the higher the voltage of the low-frequency pulse current is, the larger one stimulation is, and thus the stimulation has high intensity. Furthermore, the longer the pulse width of the low-frequency pulse current is, the longer one stimulation is, and thus the stimulation has high intensity.

Additionally, in the example illustrated in FIG. 1, the light/sound output unit 12 is disposed in the pad portion 11. For example, the light/sound output unit 12 includes the aforementioned light emitting unit disposed at a position corresponding to the position of the electrode 11a in the pad portion 11. The position corresponding to the position of the electrode 11a is a position close to the electrode 11a, and is, for example, a position closest to the electrode 11a in the surface, which can be seen from the user, of the pad portion 11 attached to the user. Moreover, the light/sound output unit 12 may include the aforementioned sound emitting unit disposed in the pad portion 11.

As just described, the light/sound output unit 12 that outputs light or sound is disposed in the pad portion 11 that supplies electrical stimulation, and thus the user can easily recognize that the light or sound being output is synchronized with the electrical stimulation. As a result, the electrical stimulation can be more effectively perceived by the user, and a physical sensation for treatment by the low-frequency pulse current can be improved.

Additionally, the electrode 11a of the pad portion 11 may include a plurality of electrodes. In this case, the light/sound output unit 12 may include a plurality of light emitting units corresponding to each of the plurality of electrodes of the pad portion 11. The plurality of light emitting units of the light/sound output unit 12 are respectively disposed in the pad portion 11 at positions corresponding to the positions of the plurality of electrodes of the pad portion 11. In addition, the plurality of light emitting units of the light/sound output unit 12 are respectively controlled by the control unit 13 to emit light in synchronization with the supply of the low-frequency pulse current by the corresponding electrodes.

Thus, light or sound is output from a location near the position where the electrical stimulation is applied in the pad portion 11. Therefore, the position to which the light or sound is output also changes in accordance with a change of the position where the electrical stimulation is applied, and the change of the position where the electrical stimulation is applied can be more effectively perceived by the user. As a result, a physical sensation for treatment by the low-frequency pulse current can be improved.

Note that in the example illustrated in FIG. 1, configuration is described such that the light/sound output unit 12 is disposed in the pad portion 11, but the light/sound output unit 12 may be disposed in the control unit 13 or may be disposed in a device different from the pad portion 11 and the control unit 13.

### Configuration of Low-Frequency Treatment Device 20 as an Example of Low-Frequency Treatment Device 10

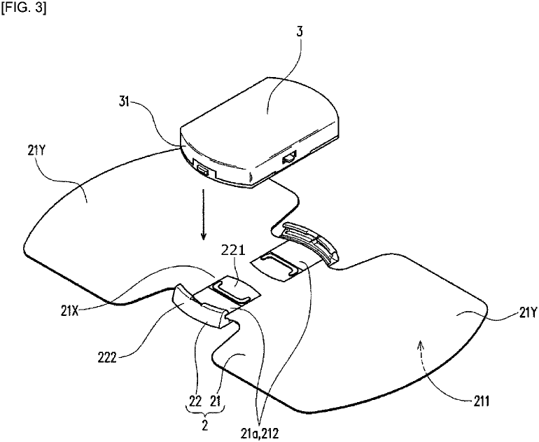

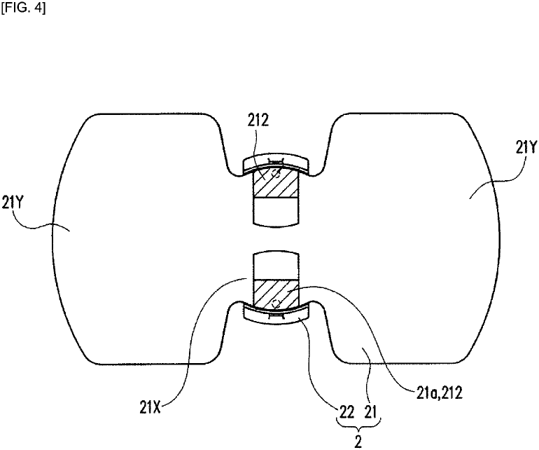

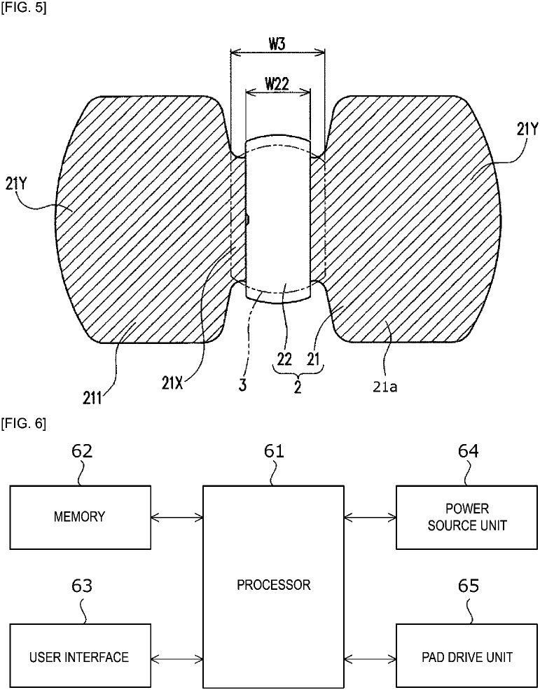

FIG. 2 is a perspective view illustrating the configuration of a low-frequency treatment device 20 that is an example of the low-frequency treatment device 10. FIG. 3 is an exploded perspective view illustrating a state where a pad portion 2 and a main body portion 3 of the low-frequency treatment device 20 are separated. FIG. 4 is a plan view illustrating the pad portion 2 of the low-frequency treatment device 20. FIG. 5 is a bottom view illustrating the pad portion 2 of the low-frequency treatment device 20.

The low-frequency treatment device 20 is a cordless type low-frequency treatment device and includes the pad portion 2 and the main body portion 3. The pad portion 2 is an example of the pad portion 11 illustrated in FIG. 1. The main body portion 3 is an example of the control unit 13 illustrated in FIG. 1.

In this example, the low-frequency treatment device 20 emits light in synchronization with the low-frequency pulse current supplied to the user, and thus allows electrical stimulation by the low-frequency pulse current to be effectively perceived by the user.

The pad portion 2 includes a pad 21 and a holder 22. The pad 21 and holder 22 are integrated in this example. “Integrated” refers to a state in which the pad 21 and the holder 22 are non-separably combined with each other in a normal usage state. However, the pad 21 and holder 22 may be separably disposed from each other.

The pad 21 is a portion to be attached to the body of the user. The pad 21 includes a conductive layer 21a that supplies a low-frequency pulse current to the user. The conductive layer 21a is an example of the electrode 11a illustrated in FIG. 1. The conductive layer 21a is exposed on at least a portion of each of front and back surfaces of the pad 21. In this example, the conductive layer 21a is exposed on the entire surface of a back surface 211 of the pad 21, which is facing to the body side of the user, and on a portion of the front surface of the pad 21.

Attaching of the pad 21 to the body of the user is performed by affixing the back surface 211 of the pad 21 to the skin of the user via a conductive gel attached to treatment portions 21Y in the range indicated by hatching in FIG. 5.

The pad 21 is formed by layering, for example, a carbon layer that is a conductor by printing on the surface of a base material made of a soft synthetic resin, and this carbon layer is the conductive layer 21a. The pad 21 has flexibility. The conductive layer 21a is provided separately for polarity (positive pole and negative pole) during energization. Note that since the pad 21 may be energized with the polarity alternately converted, the polarity is variable while the conductive layer 21a dedicated to the positive electrode and the conductive layer 21a dedicated to the negative layer are not fixedly present.

For example, as illustrated in FIG. 3, the pad 21 includes an attachment portion 21X to be attached on the holder 22, and the treatment portions 21Y each of which extends from the attachment portion 21X to at least one side and on which the conductive layer 21a is exposed.

The conductive layer 21a is also exposed on the front surface that is a surface of the attachment portion 21X of the pad 21, which faces the main body portion 3, and this exposed portion is the pad side electrode portion 212. The pad side electrode portion 212 is formed to be electrically connected to an electrode (not illustrated) of the main body portion 3.

In this example, the conductive layer 21a corresponding to one pole (for example, positive pole) is exposed at one end in a width direction of the attachment portion 21X (in an up-down direction in FIG. 4), and the conductive layer 21a corresponding to the other pole (for example, negative pole) is exposed at the other end.

The holder 22 is a portion that holds the pad 21. In this example, the holder 22 is made of a hard resin, and holds the attachment portion 21X of the pad 21 by double-sided adhesive tape. As a result, the pad 21 and the holder 22 are integrally formed.

The holder 22 includes a pad holding portion 221 that holds the attachment portion 21X of the pad 21, and wall portions 222 located at both ends of the pad holding portion 221. Note that holding of the pad 21 is not limited to a method by using double-sided adhesive tape and may be, for example, a method by heat welding or a method by using a paste or an adhesive.

The holder 22 is made of a hard resin and thus is a nonconductor. Accordingly, in a case where the pad 21 is disposed to straddle the spine on the back of the user, the holder 22 that is a nonconductor can be arranged to match the spine and the treatment portions 21Y of the pad 21 are not overlapped with the spine.

Consequently, the low-frequency pulse current is prevented from flowing through the spine and the spinal cord of the user. Accordingly, the spine and the spinal cord are prevented from being damaged by the current, and the low-frequency treatment device 20 can be safely used. In addition, it is not necessary to cover a portion of the attachment portion 21X of the pad 21, which may overlap the spine, by a separate insulating member, and thus the configuration of the pad portion 2 can be simplified.

The pad 21 is a consumable item, and the pad 21 can be attached and detached with respect to the main body portion 3 in case of replacement or the like. In this example, the holder 22 is integrally formed with the pad 21 to form the pad portion 2, and the main body portion 3 is configured to be attached and detached with respect to the holder 22. Replacement of the pad 21 is made, for example, by replacing the holder 22 as a whole.

The main body portion 3 is a portion attached to the holder 22 to apply the low-frequency pulse current to the conductive layer 21a of the pad 21. A power supply unit such as a battery and an electric circuit (a substrate) configured to form a desired low-frequency pulse current may be disposed inside the main body portion 3, and switches or a display unit may be disposed outside the main body portion 3.

Although not illustrated, electrodes to be electrically connected to the pad side electrode portions 212 of the pad portion 2 are protruded from a lower surface of the main body portion 3, which faces the holder 22. The electrodes of the main body portion 3 are separately disposed for polarities.

Here, as illustrated in FIG. 5, in a state where the holder 22 is locked to the main body portion 3, a width dimension W22 of the holder 22 is formed smaller than a width dimension W3 of the main body portion 3. The holder 22 is made of a hard resin and thus is poor in flexibility. On the other hand, since the pad 21 has flexibility, the holder 22 is formed smaller in width than the main body portion 3, and thus the flexibility of the pad 21 is less likely to be disturbed by the main body portion 3. Accordingly, since the pad 21 is easily disposed along the curved surface of the body of the user, the pad portion 2 has good fittability to the body of the user.

### Hardware Configuration of Main Body Portion 3

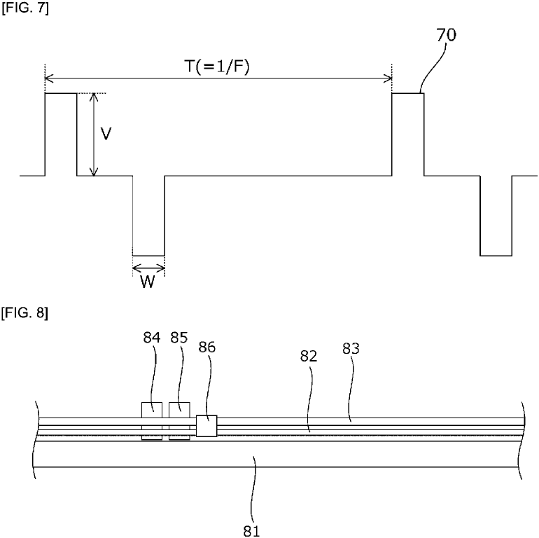

FIG. 6 is a block diagram illustrating an example of the hardware configuration of the main body portion 3. As illustrated in FIG. 6, the main body portion 3 includes, for example, a processor 61, a memory 62, a user interface 63, a power supply unit 64, and a pad drive unit 65.

The processor 61 is, for example, a processor such as a central processing unit (CPU) or a micro processing unit (MPU). The processor 61 functions as a control unit that controls the operation of components of the low-frequency treatment device 20 by reading out and executing a program stored in the memory 62. Note that the processor 61 may be a combination of multiple processors. The control unit 13 illustrated in FIG. 1 includes, for example, the processor 61.

The memory 62 is realized by a random access memory (RAM), a read-only memory (ROM), a flash memory, or the like. The memory 62 stores programs to be executed by the processor 61 or data or the like to be used by the processor 61.

The user interface 63 includes, for example, an input device that receives an operation input from the user, an output device that outputs information to the user, and the like. The input device can be implemented, for example, by a key, a remote controller, and the like. The output device can be implemented, for example, by a display, a speaker, and the like. Additionally, the input device and the output device may be implemented by a touch panel or the like. The user interface 63 is controlled by the processor 61.

Moreover, the main body portion 3 may include a communication interface instead of the user interface 63 or in addition to the user interface 63. The communication interface may be an interface for wireless communication or an interface for wired communication. The main body portion 3 is provided with the communication interface, and accordingly, for example, a command can be transmitted from another communication device such as a smart phone to the main body portion 3 to control the main body portion 3 or information of the main body portion 3 can be transmitted to another communication device.

The power supply unit 64 supplies power to each of components of the low-frequency treatment device 20. For example, an alkaline battery or a rechargeable battery such as a lithium ion battery or a nickel hydrogen battery is used as a power source, and stabilizes a battery voltage and generates a drive voltage to be supplied to each component. In addition, the power supply unit 64 is not limited to a battery, and may be configured to supply power to each component of the low-frequency treatment device 20 by using power supplied from a home power source or the like.

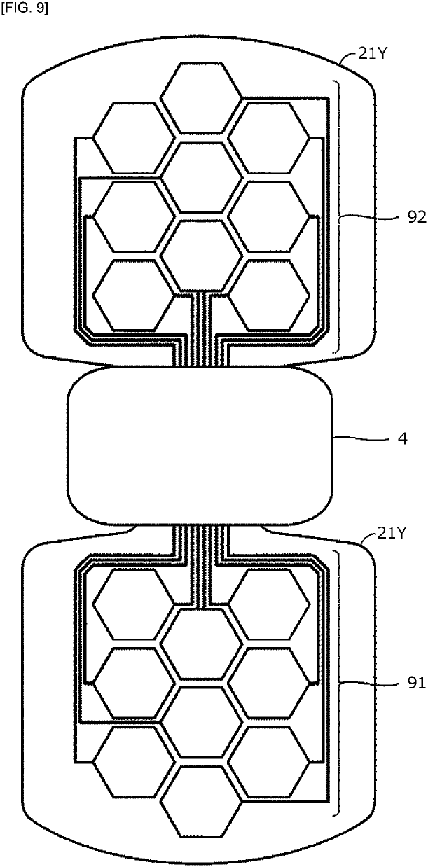

The pad drive unit 65 applies a pulse voltage to the pad 21 and thus controls supply of the low-frequency pulse current to the user by the pad 21. The pad drive unit 65 is controlled by the processor 61. In FIG. 7, the pulse voltage applied by the pad drive unit 65 of the main body portion 3 to the pad 21 will be described.

### Pulse Voltage Applied by Main Body Portion 3 to Pad 21

FIG. 7 is a diagram illustrating an example of a pulse voltage applied by the main body portion 3 to the pad 21. A pulse voltage waveform 70 illustrated in FIG. 7 is an example of a waveform of the pulse voltage applied by the pad drive unit 65 of the main body portion 3 to the pad 21.

Parameters of the pulse voltage waveform 70 include an amplitude (voltage) V, a pulse width W, and a pulse period T (pulse frequency F = 1/T). The main body portion 3 can change the treatment content for the user by changing at least one of the parameters.

Specifically, the main body portion 3 increases the power supply voltage to a predetermined voltage and adjusts the increased voltage to a voltage corresponding to the set amplitude. For example, the main body portion 3 can adjust the amplitude V of the pulse voltage at a predetermined number of levels (10-stage levels) in accordance with an instruction from the user. When receiving a setting input at a certain level from the user, the main body portion 3 generates, based on the amplitude V corresponding to the level, a treatment waveform (pulse waveform) corresponding to a treatment mode and outputs the treatment waveform to the pad side electrode portion 212 of the pad 21.

A plurality of treatment modes are prepared in advance in the low-frequency treatment device 20. The treatment modes include, for example, “massage”, “tap”, “press”, and “sweep”. The main body portion 3 applies electrical stimulation corresponding to a variety of modes from the pad 21 to the user by changing a waveform of a pulse voltage applied to the pad 21.

Also, the main body portion 3 adjusts the stimulation intensity by changing at least one of the amplitude V, the pulse width W, and the pulse period T of the pulse voltage. For example, as the amplitude V of the pulse voltage is larger, as the pulse width W is larger, or as the pulse period T (the higher the pulse frequency) is shorter, the stimulation intensity applied by the pad 21 to the user is higher.

### Pad 21 With Light Emitter

FIG. 8 is a cross-sectional view illustrating an example of the pad 21 including a light emitter.

As illustrated in FIG. 8, the pad 21 is formed in a three-layer structure of, for example, a gel layer 81, an indium tin oxide (ITO) film 82, and a polyethylene terephthalate (PET) layer 83. Also, the pad 21 is provided with a low-frequency terminal 84, an LED terminal 85, and an LED 86.

The gel layer 81 is a layer of conductive gel that is brought into contact with the body of the user. The ITO film 82 is a transparent conductive film forming the aforementioned conductive layer 21a. The PET layer 83 is a non-conductive transparent member that covers the ITO film 82. Each of the low-frequency terminal 84, the LED terminal 85, and the LED 86 is disposed to be brought in electrical contact with the ITO film 82 and exposed from the PET layer 83.

The low-frequency terminal 84 forms the aforementioned pad side electrode portion 212. The main body portion 3 applies the aforementioned pulse voltage to the low-frequency terminal 84. Also, the main body portion 3 applies a drive signal of the LED 86, which is synchronized with the pulse voltage, to the LED terminal 85. The LED 86 forms the light emitting unit included in the light/sound output unit 12 described above.

An electrode (the electrode 11a) that supplies, via the gel layer 81 to the user, a low-frequency pulse current corresponding to the pulse voltage applied from the main body portion 3 to the low-frequency terminal 84 is formed in the ITO film 82. In addition, a circuit that allows the LED 86 to emit light based on the drive signal applied from the main body portion 3 to the LED terminal 85 is formed in the ITO film 82.

The PET layer 83 is a transparent member as described above, and is a member that is visible from the user in a state where the pad 21 is attached to the body of the user. Therefore, the light emitted from the LED 86 diffuses throughout the PET layer 83 while being guided by the PET layer 83. As a result, from the user, the PET layer 83 appears to be emitting light. In this case, the PET layer 83 also forms the light emitting unit together with the LED 86.

As just described, the pad 21 includes, on an opposite surface to a surface of the gel layer 81 brought into contact with the body of the user, the PET layer 83 (an insulating layer) covering the ITO film 82, and the PET layer 83 is a transparent member that diffuses the light of the LED 86 (light emitting unit). Accordingly, for example, even when the LED 86 as a light source is small, the PET layer 83 as an insulating film is made to emit light, and thus impressive visual stimulation can be applied to the user.

Note that in the example illustrated in FIG. 8, the LED 86 is exposed from the PET layer 83 to be directly visible to the user, but the LED 86 may not be configured to be exposed. Even in such a configuration, the PET layer 83 is made to emit light, and thus impressive visual stimulation can be applied to the user.

Additionally, since the ITO film 82 is also transparent, the light emitted from the LED 86 diffuses throughout the ITO film 82 while being guided by the ITO film 82. In this case, the ITO film 82 also forms the light emitting unit together with the LED 86 and the PET layer 83. By diffusing the light in two layers of the PET layer 83 and the ITO film 82, more impressive visual stimulation can be applied to the user.

The pad 21 including a light emitter is not limited to the example illustrated in FIG. 8. For example, the pad 21 may be provided with various types of light emitters having insulating properties instead of the LED 86.

### Pad 21 in Multiple Electrode Structure

FIG. 9 is a diagram illustrating an example of the pad 21 having a multiple electrode structure. For example, as illustrated in FIG. 9, the electrode of the pad 21 may have a multiple electrode structure.

In the example illustrated in FIG. 9, the pad 21 includes electrode groups 91 and 92 in each of which a plurality of electrode portions are formed in a honeycomb geometry. The electrode group 91 is formed in one of the treatment portions 21Y, and the electrode group 92 is formed in the other of the treatment portions 21Y Each of the electrode groups 91 and 92 includes, for example, nine electrodes.

For example, the main body portion 3 (pad drive unit 65) applies a pulse voltage between two electrodes of the electrode group 91 (or the electrode group 92). Therefore, one electrode has a positive polarity and the other electrode has a negative polarity.

Since the electrode groups 91 and 92 are each formed in a honeycomb geometry, the area of the treatment portion 21Y can be efficiently utilized. However, the electrode groups 91 and 92 are not limited to the honeycomb geometry, and may be formed, for example, in a matrix form. In addition, the number of electrodes in each of the treatment portions 21Y is not limited to nine, and any number, two or more electrodes can be used.

### Light Emitting When Pad 21 Is in Multiple Electrode Structure

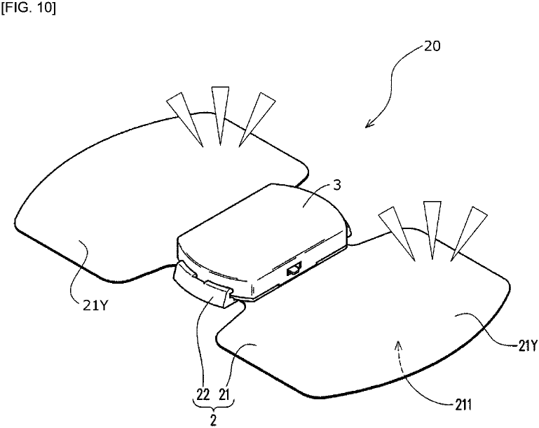

FIG. 10 is a diagram illustrating an example of light emitting in a case where the pad 21 is in the multiple electrode structure.

For example, in a case where the pad 21 is in the multiple electrode structure as illustrated in FIG. 9, a plurality of light emitting units disposed at positions of the respective electrodes in the pad 21 are provided. For example, in the configuration of the pad 21 of FIG. 8, the multiple electrode structure illustrated in FIG. 9 is formed by the ITO film 82, and the LEDs 86 are disposed near the respective electrodes of the formed multiple electrode structure. In the example illustrated in FIG. 9, the electrode groups 91 and 92 each including nine electrodes are formed, and thus eighteen LEDs 86 are disposed.

In addition, the main body portion 3 performs control for allowing the LEDs 86 near the electrodes included in the electrode groups 91 and 92 to emit light in synchronization with low-frequency pulse currents supplied from the respective electrodes. For example, at the timing of supply of the low-frequency pulse current from a certain electrode to the user, the main body portion 3 allows the LED near the electrode to emit light as illustrated in FIG. 10.

Accordingly, for example, in a configuration where the low-frequency pulse current is supplied while changing positions by using the multiple electrode structure, a location near the position stimulated by the low-frequency pulse current locally emits light. Therefore, in addition to electrical stimulation, the position of which changes, visual stimulation, the position of which changes in conjunction with the position of the electrical stimulation can be applied to the user.

As a result, as compared to a configuration in which electrical stimulation, the position of which changes is singly applied to a user, the electrical stimulation by the low-frequency pulse current is effectively perceived by the user, and thus a physical sensation for treatment by the low-frequency pulse current can be improved.

Further, a light-shielding member may be disposed in the PET layer 83 or the ITO film 82 to partition the area of each of the electrodes included in the electrode groups 91 and 92 such that the light of the LED corresponding to a certain electrode does not leak to the region corresponding to the other electrode. Thus, each area corresponding to each electrode included in the electrode groups 91 and 92 can be independently made to emit light.

Furthermore, the main body portion 3 may perform control for allowing for light emitting of not only an LED corresponding to an electrode that supplies a low-frequency pulse current, but also, for example, LEDs corresponding to electrodes around the electrode. Additionally, in this case, the main body portion 3 may change the light emission area by changing, in accordance with the intensity of a low-frequency pulse current to be supplied, the number of adjacent LEDs that are allowed to emit light.

### Color of Light, Which Changes With at Least One of Intensity and Waveform of Low-Frequency Pulse Current

The main body portion 3 may perform control for outputting light synchronized with the low-frequency pulse current and may change the color of the output light in accordance with at least one of the intensity and waveform (treatment mode) of the low-frequency pulse current. In this case, for example, an LED, the wavelength of which is variable is used as the LED 86.

For example, in a treatment mode in which a strong low-frequency pulse current is supplied to the user, the main body portion 3 outputs light such as red that applies strong visual stimulation to the user. Also, in a treatment mode in which a weak low-frequency pulse current is supplied to the user, the main body portion 3 outputs light such as pink that applies weak visual stimulation to the user. Accordingly, the difference in treatment content according to the stimulation intensity or treatment mode is visually emphasized and perceived, and thus a physical sensation for treatment by the low-frequency pulse current can be improved.

### Modified Example of Light Emitting Unit That Outputs Light Synchronized with Low-Frequency Pulse Current

The configuration is described in which the LED 86 as a light emitting unit that outputs light synchronized with the low-frequency pulse current is disposed in the pad 21, but is not limited to such a configuration. For example, a light emitting unit may be disposed in the main body portion 3 or in a device such as a smart phone that can communicate with the main body portion 3. In addition, the light emitting unit is not limited to one, and a plurality of light emitting units may be disposed.

### Low-Frequency Treatment Device 20 That Outputs Sound Synchronized with Low-Frequency Pulse Current

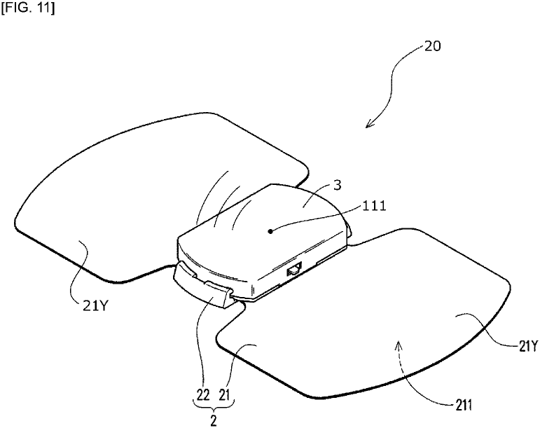

FIG. 11 is a diagram illustrating an example of the low-frequency treatment device 20 that outputs sound synchronized with a low-frequency pulse current.

Although the configuration in which the low-frequency treatment device 20 outputs light synchronized with the low-frequency pulse current is described, the low-frequency treatment device 20 may be configured to output sound synchronized with the low-frequency pulse current instead of light synchronized with the low-frequency pulse current or in addition to light synchronized with the low-frequency pulse current.

In the example illustrated in FIG. 11, the low-frequency treatment device 20 includes a speaker 111 in the main body portion 3. The main body portion 3 outputs, from the speaker 111, sound synchronized with the low-frequency pulse current supplied from the pad 21 to the user.

For example, the main body portion 3 outputs sound from the speaker 111 when the low-frequency pulse current is supplied, and does not output sound when the low-frequency pulse current is not supplied. Alternatively, the main body portion 3 may change, depending on the intensity of the supplied low-frequency pulse current, at least one of sound pressure, pitch, and tone color of the sound output from the speaker 111.

The sound output from the speaker 111 in synchronization with the low-frequency pulse current may electronic sound such as “bleep-bleep” or may be sound that evokes specific types of stimulation such as “tap-tap”, “rat-a-tat”, “yank-yank”, or “crunch-crunch”.

As just described, the low-frequency treatment device 20 outputs sound synchronized with the low-frequency pulse current. Thus, complex stimulation of electrical stimulation by the low-frequency pulse current and visual stimulation can be applied to the user. As a result, as compared to a configuration in which electrical stimulation by the low-frequency pulse current is singly applied to a user, the electrical stimulation by the low-frequency pulse current is effectively perceived by the user, and thus a physical sensation for treatment by the low-frequency pulse current can be improved.

Also, an output unit that outputs sound synchronized with the low-frequency pulse current is not limited to the speaker 111, and may be a bone conduction headphone or earphone, or the like that outputs sound to a user, for example, by bringing an oscillating object into contact with the head or neck of the user, or may output sound by generating vibration with a vibrator or the like.

### Tone Color Changing Depending on at Least One of Intensity and Waveform of Low-Frequency Pulse Current

The main body portion 3 may perform control for outputting sound synchronized with the low-frequency pulse current and also change tone color of the sound output depending on at least one of the intensity and waveform (treatment mode) of the low-frequency pulse current.

For example, in a treatment mode that supplies a strong low-frequency pulse current to a user, the main body portion 3 is configured to output sound such as a “rat-a-tat” that applies a strong auditory stimulation to the user. Additionally, in a treatment mode in which a weak low-frequency pulse current is supplied to the user, the main body portion 3 is configured to output sound such as “tap-tap” that applies weak auditory stimulation to the user. As a result, the difference in the treatment content according to the intensity of stimulation or the treatment mode is emphasized and perceived by hearing, and a physical sensation for treatment by the low-frequency pulse current can be improved.

### Modified Example of Light Emitting Unit That Outputs Sound Synchronized with Low-Frequency Pulse Current

The configuration is described in which the speaker 111 that outputs sound synchronized with the low-frequency pulse current is disposed in the main body portion 3, but is not limited to such a configuration. For example, the speaker 111 may be disposed in the pad 21 or a device such as a smart phone that can communicate with the main body portion 3.

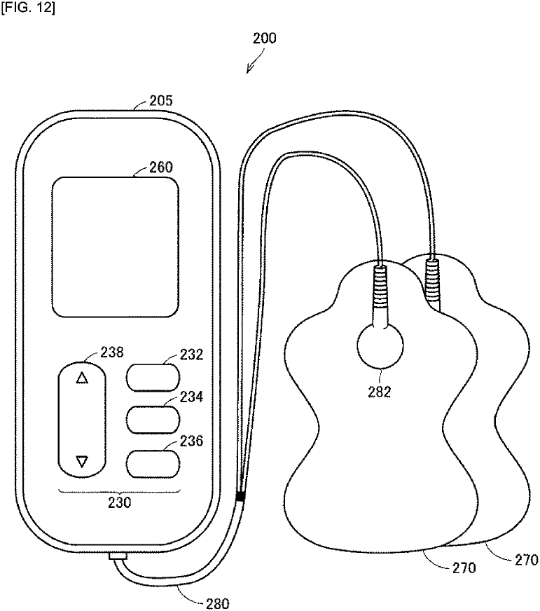

FIG. 12 is a diagram illustrating the configuration of a low-frequency treatment device 200 that is another example of the low-frequency treatment device 10.

Although the cordless type low-frequency treatment device 20 is described as an example of the low-frequency treatment device 10, but the low-frequency treatment device 10 may be the wired type low-frequency treatment device 200, for example, as illustrated in FIG. 12.

The low-frequency treatment device 200 includes a main body portion 205 of the treatment device, a pair of pads 270 configured to be attached to a treatment site, and a cord 280 that electrically connects the pads 270 to the main body portion 205. The low-frequency treatment device 200 is also a low-frequency treatment device that performs treatment of, for example, relieving shoulder stiffness of the user by supplying a low-frequency pulse in the same way as the low-frequency treatment device 20.

The pads 270 have a sheet-like shape and are configured to be attached to the user’s body. The surface on one side of the pad 270 (the surface that does not come into contact with the body) is provided with a plug that corresponds to an electrode (not illustrated) formed on the surface on the other side (the surface that comes into contact with the body). The electrode is formed from a conductive gel-like material, for example.

To connect the main body portion 205 and the pad 270, a plug 282 of the cord 280 is connected to the plug on the pad 270 and the cord 280 is inserted into the jack on the main body portion 205. Note that when the polarity of the electrode formed on one of the pads 270 is positive, the polarity of the electrode formed on the other pad 270 is negative.

The main body portion 205 includes an operation interface 230 formed of various buttons, and a display 260. The operation interface 230 includes a power button 232 for switching the power source on and off, a mode selection button 234 for selecting a treatment mode, a treatment start button 236, and an adjustment button 238 for adjusting the intensity of electrical stimulation (stimulation intensity).

Note that the operation interface 230 is not limited to the configuration described above and may have any configuration that allows the user to perform a variety of operations described below. The operation interface 230 may include, for example, other buttons, a dial, a switch, and the like.

The electrical stimulation intensity, the remaining treatment time, the treatment mode, the mounting state of the pads 270, and the like are displayed on the display 260. Various messages are also displayed on the display 260.

In the low-frequency treatment device 200, the main body portion 205 and the pads 270 include a function corresponding to the main body portion 3 and the pad 21 in the low-frequency treatment device 20. In other words, the main body portion 205 outputs from an output unit at least one of light and sound synchronized with the low-frequency pulse current supplied from the pads 270. The output unit that outputs light or sound may be disposed in the pad 270, may be disposed in the main body portion 205, or may be disposed in a device such as a smart phone that can communicate with the main body portion 205.

Additionally, in the low-frequency treatment device 200 illustrated in FIG. 12, the light emitting unit disposed in the plug 282 or the cord 280 may be configured as the output unit that outputs light synchronized with a change in a supply state of the low-frequency pulse current.

### Control Program

A control program for the low-frequency treatment device 10, the low-frequency treatment device 20, and the low-frequency treatment device 200 is stored in a non-transitory storage medium out of which the program can be read by a computer. Such a “computer-readable storage medium” includes, for example, an optical medium such as a compact disc-ROM (CD-ROM), a magnetic storage medium such as a universal serial bus (USB) memory or a memory card, etc. Additionally, such a program can also be provided by downloading via a network such as the Internet.

While various embodiments have been described with reference to the drawings, needless to say, the present invention is not limited to such examples. It will be apparent to those skilled in the art that various changes and modifications can be made within the scope of the claims, and it is understood that these are naturally belong within the technical scope of the present invention. Further, each of the components of the above-described embodiments may be combined as desired within a range that does not depart from the spirit of the present invention.

Note that the present application is based on Japanese Patent Application filed on Jul. 6, 2020 (JP 2020-116599), the content of which is incorporated herein by reference.

### REFERENCE NUMERALS LIST

2, 11 Pad portion
3, 205 Main body portion
10, 20, 200 Low-frequency treatment device
11a Electrode
12 Light/sound output unit
13 Control unit
21, 270 Pad
21X Attachment portion
21Y Treatment portion
21a Conductive layer
3 Holder
61 Processor
22 Memory
63 User interface
64 Power source unit
65 Pad drive unit
70 Pulse voltage waveform
81 Gel layer
82 ITO film
83 PET layer
84 Low-frequency terminal
85 LED terminal
86 LED
91, 92 Electrode group
111 Speaker
211 Back surface
212 Pad side electrode portion
221 Pad holding portion
222 Wall portion
230 Operation interface
232 Power button
234 Mode selection button
236 Treatment start button
238 Adjustment button
260 Display
280 Cord
282 Plug
W3, W22 Width dimension

1. A low-frequency treatment device, comprising:
a pad portion configured to be able to supply a low-frequency pulse current to a contact target; and
an output unit including atleast one of a light emitter and a sound emitter, configured to output, during supply of the low-frequency pulse current by the pad portion, at least one of light and sound synchronized with a change in a supply state of the low-frequency pulse current including stimulation intensity by the low-frequency pulse current to the contact target, wherein
at least one of the light and sound is generated based on a pulse voltage waveform for supplying the low-frequency pulse current.

2. The low-frequency treatment device according to claim 1, comprising oneor more processors configured to perform control of supply of the low-frequency pulse current by the pad portion and control, in conjunction with the control of supply of the low-frequency pulse current, of output of at least one of the light and sound by the output unit.

3. The low-frequency treatment device according to claim 1, wherein the stimulation intensity includes a frequency of the low-frequency pulse current.

4. The low-frequency treatment device according to claim 1, wherein the stimulation intensity includes a voltage of the low-frequency pulse current.

5. The low-frequency treatment device according to claim 1, wherein the stimulation intensity includes a pulse width of the low-frequency pulse current.

6. The low-frequency treatment device according to claim 1, wherein the output unit is configured to output light changing at least one of the color, brightness, and area in synchronization with a change in a supply state of the low-frequency pulse current.

7. The low-frequency treatment device according to claim 6, wherein the output unit is configured to output light having color that changes depending on at least one of intensity and waveform of the low-frequency pulse current.

8. A control method of a low-frequency treatment device including a pad portion configured to be able to supply a low-frequency pulse current to a contact target, and an output unit including at least one of a light emitter and a sound emitter, the control method comprising:
controlling the pad portion to supply the low-frequency pulse current; and
generating, based on a pulse voltage waveform for supplying the low-frequency pulse current, at least one of light and sound synchronized with a change in a supply state of the low-frequency pulse current including stimulation intensity by the low-frequency pulse current to the contact target and controlling the output unit to output, during supply of the low-frequency pulse current by the pad portion, at least one of the light and sound synchronized with a change in a supply state of the low-frequency pulse current.

9. A non-transitory recording medium in which a control program of a low-frequency treatment device is stored, the low-frequency treatment device including a pad portion configured to be able to supply a low-frequency pulse current to a contact target, and an output unit including at least one of a light emitter and a sound emitter, the control program allowing a computer to execute:
controlling the pad portion to supply the low-frequency pulse current; and
generating, based on a pulse voltage waveform for supplying the low-frequency pulse current, at least one of light and sound synchronized with a change in a supply state of the low-frequency pulse current including stimulation intensity by the low-frequency pulse current to the contact target and controlling the output unit to output, during supply of the low-frequency pulse current by the pad portion, at least one of the light and sound synchronized with a change in a supply state of the low-frequency pulse current.

10. A low-frequency treatment device, comprising:
a pad portion configured to be able to supply a low-frequency pulse current to a contact target; and
an output unit including at least one of a light emitter and a sound emitter, configured to output, during supply of the low-frequency pulse current by the pad portion, at least one of light and sound synchronized with a change in a supply state of the low-frequency pulse current including a supply position of the low-frequency pulse current in the pad portion.

11. A control method of a low-frequency treatment device including a pad portion configured to be able to supply a low-frequency pulse current to a contact target, and an output unit including at least one of a light emitter and a sound emitter, the control method comprising:
controlling the pad portion to supply the low-frequency pulse current; and
controlling the output unit to output, during supply of the low-frequency pulse current by the pad portion, at least one of light and sound synchronized with a change in a supply state of the low-frequency pulse current including a supply position of the low-frequency pulse current in the pad portion.

12. A non-transitory recording medium in which a control program of a low-frequency treatment device is stored, the low-frequency treatment device including a pad portion configured to be able to supply a low-frequency pulse current to a contact target, and an output unit including at least one of a light emitter and a sound emitter, the control program allowing a computer to execute:
controlling the pad portion to supply the low-frequency pulse current; and
controlling the output unit to output, during supply of the low-frequency pulse current by the pad portion, at least one of light and sound synchronized with a change in a supply state of the low-frequency pulse current including a supply position of the low-frequency pulse current in the pad portion.

13. A low-frequency treatment device, comprising:
a pad portion configured to be able to supply a low-frequency pulse current to a contact target; and
an output unit, including at least one of a light emitter and a sound emitter, disposed in the pad portion and configured to output, during supply of the low-frequency pulse current by the pad portion, at least one of light and sound synchronized with a change in a supply state of the low-frequency pulse current.

14. The low-frequency treatment device according to claim 13, wherein
the pad portion includes an electrode configured to be able to supply the low-frequency pulse current, and
the output unit is disposed at a position corresponding to the position of the electrode in the pad portion, and includes a light emitting unit configured to emit light in synchronization with supply of the low-frequency pulse current by the electrode.

15. The low-frequency treatment device according to claim 14, wherein
the pad portion includes a plurality of electrodes configured to be able to supply the low-frequency pulse current, and
the output unit is disposed at a position corresponding to the position of each of the plurality of electrodes in the pad portion, and includes a plurality of light emitting units configured to respectively emit light in synchronization with supply of the low-frequency pulse current by the corresponding electrodes.

16. The low-frequency treatment device according to claim 14, wherein
the pad portion includes, on a surface opposite to the contact target, an insulating layer covering the electrode, and
the insulating layer includes a transparent member configured to diffuse light of the light emitting unit.

17. The low-frequency treatment device according to claim 14, wherein the electrode is formed of a transparent conductive film configured to diffuse light of the light emitting unit.

18. A control method of a low-frequency treatment device including a pad portion configured to be able to supply a low-frequency pulse current to a contact target, and an output unit, including at least one of a light emitter and a sound emitter, disposed in the pad portion, the control method comprising:
controlling the pad portion to supply the low-frequency pulse current; and
controlling the output unit to output, during supply of the low-frequency pulse current by the pad portion, at least one of light and sound synchronized with a change in a supply state of the low-frequency pulse current.

19. A non-transitory recording medium in which a control program of a low-frequency treatment device is stored, the low-frequency treatment device including a pad portion configured to be able to supply a low-frequency pulse current to a contact target, and an output unit, including at least one of a light emitter and a sound emitter, disposed in the pad portion, the control program allowing a computer to execute:
controlling the pad portion to supply the low-frequency pulse current; and
controlling the output unit to output, during supply of the low-frequency pulse current by the pad portion, at least one of light and sound synchronized with a change in a supply state of the low-frequency pulse current.

20. A low-frequency treatment device, comprising:
a pad portion configured to be able to supply a low-frequency pulse current to a contact target; and
an output unit, including at least one of a light emitter and a sound emitter, configured to output, during supply of the low-frequency pulse current by the pad portion, sound having tone color that changes depending on at least one of intensity and waveform of the low-frequency pulse current.

21. A control method of a low-frequency treatment device including a pad portion configured to be able to supply a low-frequency pulse current to a contact target, and an output unit including at least one of a light emitter and a sound emitter, the control method comprising:
controlling the pad portion to supply the low-frequency pulse current; and
controlling the output unit to output, during supply of the low-frequency pulse current by the pad portion, sound having tone color that changes depending on at least one of intensity and waveform of the low-frequency pulse current.

22. A non-transitory recording medium in which a control program of a low-frequency treatment device is stored, the low-frequency treatment device including a pad portion configured to be able to supply a low-frequency pulse current to a contact target, and an output unit including at least one of a light emitter and a sound emitter, the control program allowing a computer to execute:
controlling the pad portion to supply the low-frequency pulse current; and
controlling the output unit to output, during supply of the low-frequency pulse current by the pad portion, sound having tone color that changes depending on at least one of intensity and waveform of the low-frequency pulse current.
